(19) United States
(12) Patent Application Publication
(10) Pub. No.: US 2005/0232886 A1
(43) Pub. Date: Oct. 20, 2005

(54) Hair treatment compositions containing itaconic acid mono-ester/acrylate copolymer and polystyrene sulfonate

(76) Inventors: Andrea Walter, Buettelborn (DE); Susanne Birkel, Darmstadt (DE); Manuela Hannich, Egelsbach (DE); Axel Kalbfleisch, Darmstadt (DE)

Correspondence Address: STRIKER, STRIKER & STENBY, 103 EAST NECK ROAD, HUNTINGTON, NY, 11743, US

(21) Appl. No.: 11/103,252

(22) Filed: Apr. 11, 2005

(30) Foreign Application Priority Data
Apr. 17, 2004 (DE) 102004018723.1

(51) Int. Cl. A61K 7/06; A61K 7/11

(52) U.S. Cl. 424/70.11

(57) ABSTRACT

The hair treatment composition contains a combination of at least one itaconic acid monoester/acrylate copolymer and at least one polystyrene sulfonate with a molecular weight in a range from 50,000 to 200,000. The itaconic acid monoester monomer or monomers are of formula CH₂═C(COOR¹)CH₂COOR², wherein one of R¹ and R² represents hydrogen and another of R¹ and R² represents —(CH₂CH₂O)ₓ—R³, x is a number between 1 and 100 and R³ is an alkyl group with 8 to 30 carbon atoms. In an especially preferred embodiment the hair treatment composition contains an itaconic acid monocetyl ester/acrylate copolymer and an itaconic acid monostearyl ester/acrylate copolymer.

### BACKGROUND OF THE INVENTION

The subject matter of the present invention is a hair treatment composition containing at least one itaconic acid monoester/acrylate copolymer and at least one polystyrene sulfonate of a selected molecular weight.

Hair gels are used in order to fix and hold human hair. Conventional hair gels usually contain a combination of gel former and hair-fixing polymer. The cosmetic hair-fixing polymers usually used for this purpose are characterized by good fixing properties, which after application more or less satisfactorily hold and fix the hair in a predetermined shape, in aqueous, alcoholic or aqueous-alcoholic media. EP 1 287 810 describes hair treatment compositions in gel form containing a combination of at least one itaconic acid monoester/acrylate copolymer and a film-forming nonionic, anionic, amphoteric or zwitterionic polymer. The known gels are still not completely satisfactory in every respect, especially regarding the sticky stage during working into the hair, the application properties on fine hair regarding load and volume effect as well as sticky feel and distributability on the hands. The so-called sticky stage plays a significant role. During and after working into the hair solvent evaporates. As a result the stickiness then strongly increases and becomes strongly fixing. An ideal gel should facilitate a suitable optimized working in time, so that the hairstyle remains sufficiently flexible and fashionable prior to gel drying and becomes strongly fixing. An ideal gel should also adhere as little as possible to the fingers and the hands but at the same time in spite of that provide good adherence to the hair. Hair styling gels are desired, which have one or more advantages without impairing good hold, e.g. loads fine hair less, provides good volume build-up in fine and heavy hair, and has an optimized sticky stage, a suitable work in time or leaves the hair feeling sticky as little as possible with good adherence.

### SUMMARY OF THE INVENTION

It is an object of the present invention to provide a hair treatment composition containing at least one itaconic acid monoester/acrylate copolymer, which has one or more of the above-described advantages without impairing good hold during hair treatment.

It has now been found that improved hairstyling gels can be provided by a hair treatment composition containing a combination of
(A) at least one copolymer of a first monomer and a second monomer, wherein the first monomer is an itaconic acid monoester of the formula CH₂═C(COOR¹)CH₂COOR², wherein one of R¹ and R² represents hydrogen and the other of R¹ and R² represents the group —(CH₂CH₂O)ₓ—R³, x is a number between 1 to 100 and R³ is an alkyl group with 8 to 30 carbon atoms, and the second monomer is an acrylate monomer; and
(B) at least one polystyrene sulfonate with a molecular weight in a range from 50,000 to 200,000.

The hair treatment composition preferably is present in gel form on an aqueous basis and preferably has a viscosity of at least 1000 mPa s at 25° C.

The copolymer (A) is preferably present in an amount of from 0.1 to 30, especially preferably from 0.5 to 15 or from 1 to 10, percent by weight. The polystyrene sulfonate is preferably present in an amount of 0.1 to 20, preferably from 0.5 to 10 or from 1 to 7, percent by weight. The hair gel according to the invention is preferably essentially free of cationic polymers, i.e. it contains either no cationic polymers or less than 0.5, preferably less than 0.1, percent by weight of cationic polymers.

The hair gel according to the invention especially provides good definition and long lasting texture to short hair and has a very beautiful luster. The gel is distributable on the hands and has a good sticky stage, which lasts sufficiently long so that the described hairstyle can be shaped, before the gel solidifies.

### Copolymer (A)

The copolymer (A) is built up from itaconic acid monoesters of the general formula CH₂═C(COOR¹)CH₂COOR², wherein one of R¹ and R² represents hydrogen and the other of R¹ and R² represents the group —(CH₂CH₂O)ₓ—R³, x is a number between 1 to 100, preferably from 10 to 40, especially preferably 20. R³ is an alkyl group with 8 to 30, preferably 12 to 20, carbon atoms, e.g. cetyl or stearyl. Cetyl is especially preferred.

The acrylate monomers of the copolymer (A) are preferably selected from the group consisting of acrylic acid, methacrylic acid and their esters, especially the acrylic acid alkyl esters and methacrylic acid alkyl esters with one to ten, especially 1 to 4, carbon atoms in the alkyl group. Suitable copolymers are, for example, acrylic acid or methacrylic acid/itaconic acid polyethoxyalkyl ester copolymers (INCI name:acrylates/Steareth-20 itaconate coplymer and acrylates/Ceteth-20 itaconate copolymer), such as marketed by the firm National Starch, U.S.A. under the trademark Structure® 2001 and Structure® 3001. The acid groups in the polymers used are preferably neutralized by organic or inorganic bases to the extent of 50 to 100%. Suitable neutralization agents are primary or secondary amines, especially aminoalkanols with preferably from 1 to 10 carbon atoms and 1 to 3 hydroxy groups, such as e.g. aminomethyl propanol (AMP), triethanol amine, tetrahydroxypropylethylenediamine or monoethanolamine, but also ammonia, sodium hydroxide, potassium hydroxide, among others.

In an especially preferred embodiment the composition according to the invention contains at least one first and at least one second copolymer each comprising an itaconic acid monoesters of the formula CH₂═C(COOR¹)CH₂COO—(CH₂CH₂O)ₓ—R³, x is a number between 10 to 40 and R³ of the first copolymer is a cetyl group and R³ of the second copolymer is a stearyl group. The weight ratio of the first copolymer (cetyl derivative) to the second copolymer (stearyl derivative) is preferably greater than or equal to 1:1, especially from 2:1 to 10:1, especially preferably from 3:1 to 5:1. This composition with the two itaconate acid monoester/acrylate copolymers is characterized by a special long time stability of the especially advantageous consistency and Theological properties.

### Polystyrene Sulfonate (B)

The polystyrene sulfonate is made by polymerization of styrene, sulfonation of the obtained polystyrene and salt formation of the obtained poly(styrene sulfonic acid) with a suitable base, e.g. sodium hydroxide. A typical formula is —[CH(C₆H₄—SO₃⁻A⁺]ₙ—, wherein n is a natural number greater than 0, which gives the polymerization degree and is selected so that the molecular weight of the polymer is in a range from 50,000 to 200,000, preferably 80,000 to 180,000. A⁺ is a suitable cation, especially a metal cation, preferably an alkali metal or alkaline earth metal cation, e.g. sodium. A suitable commercial product is e.g. FLEXAN® 130 or FLEXAN® II with a molecular weight of about 130,000, a glass transition temperature of about 112° C. and a viscosity in a range from 50 to 250 cps (30% aqueous solution, RTV Brookfield Viscometer, 20 rpm, Spindel Nr. 2, 25° C.).

In preferred embodiments the hair treatment composition of the invention can include at least one additional hair-fixing polymer different from polymer (A) and polymer (B). The hair-fixing polymer can be nonionic, anionic, zwitterionic or amphoteric, however preferably anionic or nonionic. It can be a synthetic or natural polymer. The term “natural polymer” means any polymer of natural origin or any polymer of natural origin that has been chemically modified. Those polymers are particularly preferred, which have sufficient solubility in water, alcohol or alcohol/water mixtures, in order to be present in completely dissolved form in the compositions according to the invention. “Hair-fixing polymers” are understood to be those polymers, which, when used in an amount of from 0.01 to 5% in an aqueous, alcoholic or aqueous-alcoholic solution or dispersion, are in a position to deposit a polymer film on the hair and to fix the hair. “Hair-fixing polymers” are especially those polymers listed under the function “Hair Fixatives” in the International Cosmetic Ingredient Dictionary and Handbook.

Suitable synthetic non-ionic polymers include homopolymers or copolymers, which are built up from at least one of the following monomers: vinylpyrrolidone, vinylcaprolactam, vinyl esters, such as vinyl acetate, vinyl alcohol, acrylamide, methacrylamide, alkyl- and dialkylacrylamide, alkyl- and dialkylmethacrylamide, dialkylaminoalkylmethacrylamide, dialkylamino-alkylacrylamide, alkylacrylate, alkylmethacrylate, propylene glycol or ethylene glycol, wherein the alkyl groups in these monomers preferably have from one to seven carbon atoms, especially preferably from one to three carbon atoms. For example, homopolymers of vinyl caprolactam, of vinyl pyrrolidone or of N-vinylformamide, are especially suitable. Additional suitable synthetic hair-fixing polymers are, e.g., copolymerizates of vinyl pyrrolidone and vinyl acetate, terpolymers of vinyl pyrrolidone, vinyl acetate and vinyl propionate, terpolymers of vinyl pyrrolidone, vinyl caprolactam and dialkylamino-alkyl(meth)acrylate, terpolymers of vinyl pyrrolidone, vinyl caprolactam and dialkylaminoalkyl(meth)acrylamide, polyacrylamide, polyvinyl alcohols, and hair-fixing polyethylene glycol/polypropylene glycol copolymers. Polyvinylpyrrolidone and polyvinylpyrrolidone/vinyl acetate copolymers are especially preferred.

Suitable anionic hair-fixing polymers include natural or synthetic homo- or copolymers with monomer units containing acid groups, which are copolymerizable with comonomers, if necessary, which contain no acid groups. The acid groups preferably include —COOH, —SO₃H, —OSO₃H, —OPO₂H and —OPO₃H groups, of which the carboxylic acid groups are especially preferred. The acid groups of these anionic polymers can be present in unneutralized, partially or completely neutralized form. They are preferably present in the composition according to the invention in anionic or neutralized form that is 50% to 100% neutralized. The above-mentioned neutralizing agents may be used to neutralize the acid groups. Suitable monomers include unsaturated, radically polymerizable compounds, which have at least one acid group, especially carboxyvinyl monomers. Suitable monomers containing acid groups include, e.g., acrylic acid, methacrylic acid, crotonic acid, maleic acid and/or maleic acid anhydride or their monoesters, aldehydocarboxylic acids or ketocarboxylic acids.

Comonomers not substituted with acid groups include, e.g., acryl amide, methacrylamides, alkyl and dialkylacrylamides, alkyl and dialkylmethacrylamides, alkylacrylates, alkylmethacrylates, vinyl caprolactone, vinyl pyrrolidone, vinyl esters, vinyl alcohol, propylene glycol or ethylene glycol, amine-substituted vinyl monomers, such as dlalkylamirnoalkylacrylates, dialkylaminoalkylmethacrylates, monoalkylaminoalkylacrylates and monoalkylaminoalkylmethacrylates, in which the alkyl groups of these monomers preferably contain one to seven carbon atoms, especially preferably from one to three carbon atoms.

Suitable anionic polymers especially include copolymers of acrylic acid or methacrylic acid with monomers selected from the group consisting of acrylic acid esters, methacrylic acid esters, acryl amides, methacrylamides and vinylpyrrolidones, homopolymers of crotonic acid and copolymers of crotonic acid with monomers selected from the group consisting of vinyl esters, acrylic acid esters, methacrylic acid esters, acrylamides and methacrylamides. A suitable natural polymer is, for example, shellac.

Preferred anionic polymers include cross-linked or uncross-linked vinyl acetate/crotonic acid copolymers. Similarly partially esterified copolymers between vinyl methyl ether and maleic acid anhydride are also preferred. Additional suitable anionic polymers include, e.g., terpolymers of acrylic acid, alkyl acrylate and N-alkylacrylamide, especially acrylic acid/ethyl acrylate/ N-t-butylacrylamide terpolymer, terpolymers of vinyl acetate, crotonate and vinyl alkanoate, especially vinyl acetate/crotonate/vinyl neodecanoate copolymers.

Suitable film-forming amphoteric polymers include those polymers, which contain both basic or cationic groups, especially primary, secondary and tertiary amine groups, besides acidic or anionic groups, as additional functional groups. For example, suitable amphoteric polymers are copolymers made from alkylacrylamides (especially octylacrylamide), alkylaminoalkylmethacrylates (especially t-butylamino-ethylmethacrylate), and two or more monomers selected from the group consisting of acrylic acid, methacrylic acid and their esters, in which the alkyl groups have from one to four carbon atoms and at least one of the monomers has an acid group, such as those which are obtainable under the trademarks AMPHOMER® and AMPHOMER® LV-71 of National Starch, U.S.A.

Further examples of suitable copolymers include copolymers of acrylic acid, methacrylate and methacrylamideopropyltrimethylammonium chloride (INCI: polyquaternium-47), copolymers made from acrylamidopropyltrimethyl ammonium chloride and acrylates or copolymers made from acrylamide, acrylamidopropyltrimethylammonium chloride, 2-amidopropylacrylamide sulfonate and dimethylaminopropylamine (INCI: Polyquaternium-43). Suitable polymers made with monomers carrying betaine groups, such as copolymers of methacryloylethylbetaine and two or more monomers made from acrylic acid or their simple esters, known under the INCI designation methacryloyl ethyl betaine/acrylates copolymer.

The composition according to the invention is preferably provided in an aqueous, alcoholic or an aqueous-alcoholic medium with preferably at least 10 percent by weight water and preferably up to a maximum amount of 40 percent by weight alcohol. Lower univalent or multivalent alcohols suitable for cosmetic purposes and having from one to four carbon atoms, such as ethanol and isopropanol, may be used as the alcohol. The composition according to the invention has a pH of greater than 7, preferably at least 7.3.

In a preferred embodiment the gel contains polyhydric alcohols for further Improvements in luster, preferably those with 2 to 5 carbon atoms and with 2 to 5 hydroxy groups in an amount of 0.1 to 15 percent by weight, preferably from 1 to 10 percent by weight. Glycerol, ethylene glycol and propylene glycol, especially 1,2-propylene glycol and 1,2-pentandiol, are especially preferred.

The viscosity of the gel preferably amounts to from 1000 to 100,000 mPa.s, especially preferably from 2000 to 50,000 mPa.s, and most preferably from 2,500 to 15,000 mpa.s, measured as dynamic viscosity with a Haake VT-550 Rheometer, measurement body SV-DIN at a temperature of 25° C. and a shear rate of 50 s⁻¹.

The compositions according to the invention can also contain conventional additive ingredients suitable for hair treatment compositions. These additive ingredients include, e.g. wetting agents or emulsifiers, from the classes of nonionic, anionic, cationic or amphoteric surfactants, such as fatty alcohol sulfates, alkyl benzene sulfonates, alkyltrimethylammonium salts, alkyl betaines, ethoxylated fatty alcohols, ethoxylated fatty acid glycerides, in an amount of from 0.1 to 15 percent by weight; moisturizers; perfume oils in an amount of 0.1 to 1.0 percent by weight; turbidity-imparting agents, such as ethylene glycol distearate, in an amount of about 0.2 to 5.0 percent by weight; pearlescence-imparting agents, such as a mixture of fatty acid monoalkylolamide and ethylene glycol distearate, in an amount of from about 1.0 to 10 percent by weight; bactericidal and fungicidal agents, for example, 2,4,4 trichloro-2-hydroxydiphenyl ether or methyl chloroisothiazolone, in an amount of from 0.01 to 1.0 percent by weight; thickeners, such as coconut fatty acid diethanol amides, in an amount of about 0.2 to 3.0 percent by weight; buffer substances, such as sodium citrate or sodium phosphate, in an amount of from 0.1 to 1.0 percent by weight; colorants, for example, fluorescein sodium salt, in an amount of about 0.1 to 1.0 percent by weight; care materials, such as plant and vegetable extracts, protein and silk hydrolyzates, lanolin derivative compounds, in an amount of 0.1 to 5 percent by weight; physiologically compatible silicone derivative compounds, such as volatile or non-volatile silicone oils or high-molecular-weight siloxane polymers, in an amount of from 0.05 to 20 percent by weight; light protective materials, antioxidants, radical trapping agents, anti-flaking active ingredients, in an amount of about 0.01 to 4 percent by weight; fatty alcohols; luster-imparting agents; vitamins; softening agents; combability-improving agents, lubricating agents and anti-foaming agents.

The composition according to the invention is preferably in the form of a clear, transparent or at least translucent gel. The gel can be colored or colorless.

The composition according to the invention is characterized by Theological properties, which particularly manifest themselves in an attractive appearance and pleasant haptic properties. The composition is easily distributed on the hair. When formulated as a hair treatment agent it does not load the hair and is especially good for fine hair. It provides a sufficient hold for the hairstyle, without adhering to the hair or loading it. Fine hair becomes full and voluminous. As a hair fixing gel the hair treatment composition provides improved fixing properties and especially a clearly better luster.

In a special embodiment the gel according to the invention is suitable for simultaneously fixing and temporarily coloring hair. It contains additionally at least one temporary hair colorant. A “temporary hair coloring” means a color change of human hair, which lasts until the next hair washing and which can be removed again by washing the with conventional shampoos. The pigments are, preferably, contained in the composition in an amount of from 0.01 to 25 percent by weight, especially preferably in an amount of 5 to 15 percent by weight. The pigments are preferably micro-pigments, not nano-pigments. The preferred particle size amounts to from 1 to 200 μm, especially from 3 to 150 μm, especially preferably from 10 to 100 μm.

The pigments are practically insoluble coloring agents and can be inorganic or organic. Also inorganic-organic mixed pigments may be used. Inorganic pigments are preferable. The advantage of the inorganic pigments is their outstanding light-resistance, weather-resistance and temperature-resistance. The inorganic pigments can be of natural origin, for example chalk, ocher, umber, green earth, burnt siena or graphite. The pigments can be white pigments, such as titanium dioxide or zinc oxide; black pigments, such as iron oxide black; fancy or multi-colored pigments, such as ultramarine or iron oxide red; lustrous pigments, metal effect pigments, pearlescent pigments as well as fluorescent or phosphorescent pigments. Preferably at least one pigment is a colored, non-white pigment. Metal oxides, metal hydroxides and metal oxide hydrates, mixed phase pigments, sulfur-containing silicates, metal sulfides, complex metallo-cyanides, metal sulfates, metal chromates and metal molybdates and metals themselves (bronze pigments) are suitable. In particular, titanium dioxide (C.I. 77891), black iron oxide (C.I. 77499), yellow iron oxide (C.I. 77492), red and brown iron oxide (C.I. 77491), manganese violet (C.I. 77742), ultramarine (sodium aluminum sulfosilicate, C.I. 77007, Pigment Blue 29), chromium oxide hydrate (C.I. 77289), Iron Blue (Ferric ferrocyanide, C.I. 77510) and carmine (cochineal), are all suitable pigments.

Pigments based on mica and/or isinglass, which are coated with a metal oxide or metal oxychloride, such as titanium dioxide or bismuth oxychloride and if necessary other color-imparting materials, such as iron oxides, iron blue, ultramarine, carmine, etc, whose colors can be modified by changing the thickness of the coating, are especially preferred. Pigments of this sort are marketed, for example, under the trademark, Rona®, Colorona®, Dichrona® and Timiron® by the firm, Merck, Germany.

Organic pigments are, for example, the natural pigments, Sepia, gamboge, charcoal, Kasseler brown, indigo, chlorophyl and other plant pigments. Synthetic organic pigments include, for example, azo pigments, anthraquinone pigments, indigo pigments, dioxazine, quinacridone, phthalocyanine isoindolinone pigments, perylene pigments, perinone pigments, metal complex pigments, alkali blue pigments and diketopyrrolopyrrole pigments.

A preferred embodiment of the hair treatment composition contains
(A) from 0.5 to 15 percent by weight of a copolymer, which comprises a first monomer, namely an ethoxylated itaconic acid monocetyl ester with an ethoxylation degree of 10 to 40, and a second monomer, namely an acrylate monomer; and
(B) from 0.5 to 7 percent by weight of a polystyrene sulfonate with a molecular weight of 80,000 to 180,000.

This preferred embodiment is in the form of an aqueous gel and has a pH of 6 to 9.

The following examples illustrate the above-described invention in more detail, but the details in these examples should not be considered as limiting the claims appended hereinbelow. The polymer content given in the examples relates to the solids content, unless otherwise designated.

### EXAMPLES

### Example 1

### Hair Gel

| | |
|---|---|
| 10 g | STRUCTURE ® 3001* |
| 5 g | FLEXAN ® II** |
| 5 g | 1,2-propylene glycol |
| 0.2 g | PEG-40 hydrogenated castor oil |
| 0.2 g | Perfume |
| To 100 g | Water |

*acrylates/ceteth-20 Itaconate copolymer, 30% in water
**sodium polystyrene sulfonate, Molecular weight about 130,000.

The pH value is adjusted with aminomethylpropanol to a pH in a range from 6 to 8.

Consistency: Gel, easily distributed, good washability

### Example 2

### Hair Gel

| | |
|---|---|
| 17 g | STRUCTURE ® 3001* |
| 4 g | FLEXAN ® II** |
| 2 g | Glycerol |
| 0.2 g | Perfume |
| 0.2 g | PPG-1-PEG-9 lauryl glycol ether |
| 0.4 g | TIMIRON ® pigment |
| To 100 g | Water |

*acrylates/ceteth-20 Itaconate copolymer, 30% in water
**sodium polystyrene sulfonate, Molecular weight about 130,000.

The pH value is adjusted to 6 to 8.

Consistency: Gel, easily distributed, good washability

### Example 3

### Hair Gel

| | |
|---|---|
| 8 g | STRUCTURE ® 3001* |
| 7 g | FLEXAN ® II** |
| 10 g | 1,2-propylene glycol |
| 0.2 g | Perfume |
| 0.2 g | Oleth-20 |
| 0.065 g | Dyes (D + C Violet No. 2, |
| | D + C Red No. 33, COLORONA ® |
| | pigment |
| To 100 g | Water |

*acrylates/ceteth-20 Itaconate copolymer, 30% in water
**sodium polystyrene sulfonate, Molecular weight about 130,000.

The pH value is adjusted to 6 to 8.

Consistency: Gel, easily distributed, good washability

### Example 4

### Hair Gel

| | |
|---|---|
| 15 g | STRUCTURE ® 3001* |
| 9 g | FLEXAN ® II** |
| 5 g | 1,2-propylene glycol |
| 0.15 g | Perfume |
| 0.15 g | PEG-40 hydrogenated castor oil |
| 0.3 g | COLORONA ® pigment |
| q.s. | Coloring dye |
| To 100 g | Water |

*acrylates/ceteth-20 Itaconate copolymer, 30% in water
**sodium polystyrene sulfonate, Molecular weight about 130,000.

The pH value is adjusted to 6 to 8.

Consistency: Gel, easily distributed, good washability

### Example 5

### Hair Gel

| | |
|---|---|
| 10 g | STRUCTURE ® 3001* |
| 9 g | FLEXAN ® II** |
| 5 g | 1,2-Pentandiol |
| 0.15 g | Perfume |
| 0.15 g | PPG-1-PEG-9 lauryl glycol ether |
| 0.3 g | TIMIRON ® pigment |
| q.s. | Dye compounds |
| To 100 g | Water |

*acrylates/ceteth-20 Itaconate copolymer, 30% in water
**sodium polystyrene sulfonate, Molecular weight about 130,000.

The pH value is adjusted to 6 to 8.

Consistency: Gel, easily distributed, good washability

TABLE I
COMPOSITIONS OF A TESTED
EXAMPLE OF THE COMPOSITION
ACCORDING TO THE INVENTION
AND PRIOPR ART COMPARATIVE
COMPOSITIONS

| Ingredient | A | B | C | D |
|---|---|---|---|---|
| STRUCTURE ® 3001* | 10 | 10 | 10 | 10 |
| FLEXAN ® II** | 5 | — | — | — |
| AMP-acrylates/allyl methacrylate | — | 5 | — | — |
| copolymer | | | | |
| AMPHOMER ®*** | — | — | 5 | — |
| Vinyl pyrrolidone/vinyl | — | — | — | 5 |
| acetate copolymer | | | | |
| 1,2-propylene glycol | 3.6 | 3.6 | 3.6 | 3.6 |
| PEG-40 hydrogenated castor oil | 0.2 | 0.2 | 0.2 | 0.2 |
| PHB methyl ester | 0.4 | 0.4 | 0.4 | 0.4 |
| Water | To 100 | To 100 | To 100 | To 100 |

*acrylates/ceteth-20 Itaconate copolymer, 30% in water
**sodium polystyrene sulfonate, Molecular weight about 130,000
***octylacrylamide/acrylates/butylaminoethylmethacrylate copolymer

The pH values are adjusted with aminomethylpropanol to a value in a range of 6 to 8.

The gel A according to the invention and the gels B, C and D not according to the invention were evaluated qualitatively by six test persons in regard to consistency, sticky stage and feel on the hands as well as distributability. The evaluation scale was numerical, ranging from 1 (very good) to 5 (very poor). The resulting numerical values for the tested compositions appear in the following Table 11 and are rounded average values.

TABLE II
COMPARISON OF RELEVANT
PROPERTIES OF AN EXAMPLE OF
THE COMPOSITION OF THE INVENTION
WITH PROPERTIES OF THE
COMPARATIVE COMPOSITIONS OF THE PRIOR ART

| Property | A | B | C | D |
|---|---|---|---|---|
| Consistency | 2 | 4 | 3 | 3 |
| Sticky stage | 2 | 3 | 3 | 3 |
| Feel | 2 | 2 | 3 | 2 |
| Distributability | 1.5 | 2 | 3 | 2 |

The gel A according to the invention clearly has significant properties that are unexpectedly better than any of the comparative compositions of the prior art. The gel A is clearly better in the sticky or adhesive stage and is better than any other individual gel. Also the distributability is clearly unexpectedly improved in relation to all the exemplary gels of the prior art.

### Example 6

### Styling Gel

| | |
|---|---|
| 8 g | STRUCTURE ® 3001 (acrylates/ceteth-20 |
| | Itaconate copolymer, 30% in water) |
| 5 g | FLEXAN ® II (sodium polystyrene sulfonate, |
| | Molecular weight about 130,000) |
| 2 g | STRUCTURE ® 3001 (acrylates/steareth-20 |
| | Itaconate copolymer, 30% in water) |
| 1.75 g | Aminomethylpropanol (95%) |
| 0.25 g | CARBOMER |
| To 100 g | Water |

The disclosure in German Patent Application 10 2004 018 723.1 of Apr. 17, 2004 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a hair treatment composition containing itaconic acid monoester/acrylate copolymer and polystyrene, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A hair treatment composition containing a combination of
at least one copolymer of a first monomer and a second monomer, wherein the first monomer is an itaconic acid monoester of the formula CH₂═C(COOR¹)CH₂COOR², wherein one of R¹ and R² represents hydrogen and another of R¹ and R² represents —(CH₂CH₂O)ₓ—R³, x is a number between 1 and 100 and R³ is an alkyl group with 8 to 30 carbon atoms, and the second monomer is an acrylate monomer; and
at least one polystyrene sulfonate with a molecular weight of from 50,000 to 200,000.

2. The hair treatment composition as defined in claim 1, wherein said at least one copolymer is present in an amount of 0.1 to 30 percent by weight.

3. The hair treatment composition as defined in claim 1, wherein said at least one polystyrene sulfonate is present in an amount of 0.1 to 20 percent by weight.

4. The hair treatment composition as defined in claim 1, wherein said x is a number between 10 and 40 and said R³is an alkyl group with 12 to 20 carbon atoms.

5. The hair treatment composition as defined in claim 1, wherein said second monomer is acrylic acid, methacrylic acid, an acrylic acid alkyl ester containing an alkyl group with one to ten carbon atoms or a methacrylic acid alkyl ester containing an alkyl group with one to ten carbon atoms.

6. The hair treatment composition as defined in claim 1, wherein said molecular weight of said at least one polystyrene sulfonate is from 80,000 to 180,000.

7. The hair treatment composition as defined in claim 1, substantially free of cationic polymers.

8. The hair treatment composition as defined in claim 1, further comprising at least one alcohol and wherein said at least one alcohol is selected from the group consisting of monohydric alcohols with one to four carbon atoms and polyhydric alcohols with 2 to 5 carbon atoms.

9. The hair treatment composition as defined in claim 1, further comprising at least one hair-fixing polymer and wherein said at least one hair-fixing polymer is nonionic, anionic, zwitterionic or amphoteric.

10. The hair treatment composition as defined in claim 9, wherein said at least one hair-fixing polymer is polyvinyl pyrrolidone, vinyl pyrrolidone/vinyl acetate copolymer, vinyl acetate/crotonic acid copolymer, a terpolymer of vinyl acetate, crotonate and vinyl alkanoate, a partially esterified copolymer of vinyl methyl ether and maleic acid anhydride, a copolymer of acrylic acid or methacrylic acid with an alkyl acrylate and/or an N-alkylacrylamide or a copolymer of alkylacrylamide, alkylaminoalkylmethacrylate and two or more monomeric compounds;
wherein said monomeric compounds are selected from the group consisting of acrylic acid, methacrylic acid, C₁— to C₄-alkyl esters of acrylic acid and C₁— to C₄-alkyl esters of methacrylic acid and at least one of the monomeric compounds has an acid group.

11. The hair treatment composition as defined in claim 1, in the form of an aqueous gel having a pH of 6 to 9; and
wherein said first monomer is an ethoxylated itaconic acid monocetyl ester with an ethoxylation degree of 10 to 40 and said molecular weight of said at least one polystyrene sulfonate is in a range from 80,000 to 180,000; and
wherein said at least one copolymer is present in an amount of 0.5 to 15 percent by weight and said at least one polystyrene sulfonate is present in an amount of 0.5 to 7 percent by weight.

12. The hair treatment composition as defined in claim 1, further comprising at least one hair-coloring pigment, in order to simultaneously fix and temporarily color hair.

13. The hair treatment composition as defined in claim 12, containing from 0.01 to 25 percent by weight of said at least one hair-coloring pigment and wherein said at least one hair-coloring pigment is a metal oxide pigment, ultramarine, a lustrous pigment, a metal-effect pigment, a pearlescent pigment, a fluorescent pigment, a phosphorescent pigment, a metal hydroxide pigment, a metal oxide hydrate pigment, a mixed phase pigment, a sulfur-containing silicate pigment, a metal sulfide pigment, a complex metallo-cyanide pigment, a metal sulfate pigment, a metal chromate pigment, a metal molybdate pigment, a bronze pigment, carmine and/or a mica-based pigment, which is coated with a metal oxide or a metal oxychloride and optionally an additional coloring substance, so that a color of the mica-based pigment is determined by variation of coating thickness.

14. The hair treatment composition as defined in claim 13, wherein said additional color substance is an iron oxide, iron blue, ultramarine and/or carmine.

15. The hair treatment composition as defined in claim 1, wherein said at least one copolymer comprises a first copolymer and a second copolymer, each of which is of the formula: CH₂═C(COOR¹)CH₂COO—(CH₂CH₂O)ₓ—R³, wherein x is a number between 10 to 40 and R³ of the first copolymer is a cetyl group and R³ of the second copolymer is a stearyl group.

16. The hair treatment composition as defined in claim 14, wherein said first copolymer and said second copolymer are present in a weight ratio greater than or equal to 1:1.
